(19) United States
(12) Patent Application Publication
(10) Pub. No.: US 2011/0230732 A1
(43) Pub. Date: Sep. 22, 2011

(54) System utilizing physiological monitoring and electronic media for health improvement

(75) Inventors: Carl Frederick Edman, San Diego, CA (US); Darrel Dean Drinan, San Diego, CA (US); Naresh Chandra Bhavaraju, San Diego, CA (US)

(73) Assignee: PhiloMetron, Inc., San Diego, CA (US)

(21) Appl. No.: 12/807,835

(22) Filed: Sep. 14, 2010

Related U.S. Application Data
(60) Provisional application No. 61/276,603, filed on Sep. 14, 2009.

(51) Int. Cl. A61B 5/00

(52) U.S. Cl. 600/301

(57) ABSTRACT

The present invention generally relates to the use of physiological monitoring to enable an individualized response, e.g. feedback, recommendations, rewards or guidance to be presented to the individual within the context of electronic media. Specifically, the response is preferably based upon measured physiological data obtained while the individual is not directly involved with the electronic media and is preferably delivered in the context of an electronic game or interactive social activity program.

### CROSS REFERENCE TO RELATED PATENTS

This application claims priority under U.S.C. Section 119(e) to provisional application No. 61/276,603, filed on Sep. 14, 2009.

### FEDERALLY SPONSORED RESEARCH OR DEVELOPMENT

Not Applicable

### BACKGROUND OF THE INVENTION

Rising health costs are a growing problem within society, and are driven in large part by increased obesity. Obesity raises the likelihood of developing chronic diseases such as Type II diabetes, cardio-vascular disease, and kidney disease, resulting in dramatic and, to a large extent, preventable health care costs. The origin of excess weight in the majority of overweight individuals is from poor eating habits driven by dysfunctional behavior patterns. There is therefore a need to provide effective tools for behavior guidance enabling the management of obesity, including tools for youth to limit occurrence of adverse events later on in life.

In addition to obesity, there exist other health needs that would benefit from behavioral changes or guidance. For example, the elderly in general may benefit from increased physical activity or a change in eating habits, e.g. greater hydration, to improve their health. There is a need for behavioral change to encourage these behaviors especially outside of direct instruction or supervision. Likewise individuals recovering from surgery also may benefit from methods that encourage positive behavioral changes, e.g. behaviors that may be followed outside of rehabilitation sessions to speed recovery. Unfortunately, simple instructions or recommendations frequently fail to influence behavior in desired fashions due to either the lack of personalization of the guidance or the format/delivery of the message. As one method to overcome these deficiencies, the use of animated computer activities to encourage healthful behaviors is one approach increasingly utilized.

Numerous examples exist of computer games or visualizations coupled with physical activity for the purpose of improved health. For example, Neff et al. (U.S. patent application Ser. No. 11/495,229) teach the use of exercise equipment coupled with video displays to provide instructional information regarding activity and health. They also teach the coordination of video displays to physical activity conducted. However, they fail to teach methods for monitoring behavior or activity associated with health while not directly engaged with the exercise equipment thereby failing to account for those periods of time when the user is not directly engaged with the system.

Likewise, Southard et al. (U.S. patent application Ser. No. 10/390,068) speak to the use of an electronic game employing physical activity monitoring or data inputted by an adult, to reward individuals for activity and thereby serves to encourage behavior aimed at reducing obesity. However, this approach does not teach the use of activity outside of the game environment or provide a means to directly monitor this activity and thereby accurately reward the individual. Furthermore, this input requires the intervention of a third party, e.g. a parent, to encourage behavioral change outside of the direct game experience.

Wessel (U.S. patent application Ser. No. 11/617,591) teaches the use of rewards or incentives to encourage the monitoring of physiological parameters and the use of reward tables in conjunction with historical physiological data to gauge reward level. However, the disclosure does not teach the use of incorporating the encouragement or rewards within the context of a different format, e.g. a game, to facilitate use by the individual nor does it teach the use of monitoring while not engaging the elements of the invention. In addition, Wessel does not teach the use of other formats/content other than the direct reward/incentive messages to convey success to the individual. Therefore interaction between the individual and the invention may be transitory in nature, i.e. restricted to that period of time when the incentive message is conveyed to the individual, and therefore is potentially lessened in effectiveness.

In short, there remains a need for a monitoring of an individual while not directly engaged with an electronic media, e.g. game, etc., whereby such monitoring is provided to the electronic media and thereby alters the media content in a fashion reflective of one or more measured parameters.

### SUMMARY OF THE INVENTION

The present invention claims the use of monitored physiological data collected while an individual is not directly engaged with electronic media, the analysis of this data for the determination of at least one physiological parameter, and then the incorporation of at least some portion of this analysis into at least one form of electronic media whereby the electronic media's presentation to the individual is modified in some form by the analysis of the measured physiological data.

The present invention also claims at least one monitor having at least one sensor to measure at least one physiological parameter of an individual, at least one electronic device able to receive data from at least one monitor, at least one evaluator contained substantially within the electronic device capable of evaluating the measured physiological parameter data of the individual and generating a response to said analysis and at least one form of electronic media for communicating said response to the individual and is in communication with the electronic device.

In preferred embodiments of the present invention, the measured physiological parameter or parameters are related to the metabolic status and/or health of the individual. In such embodiments, one or more sensors enabling the determination of body composition change and/or activity are preferably utilized.

In preferred embodiments of the present invention, the monitor is affixed to the individual for an extended period of time and communicates at least a portion of the measured data or analysis of the measured data by wireless means to at least one electronic device which contains at least one evaluator. The evaluator in turns communicates at least one response to electronic media.

In preferred embodiments of the present invention, the electronic media is an electronic game contained which is adjusted to the analyzed data by awarding points to the individual within the gaming context. Such points may be utilized to alter the playing capabilities of one or more avatars contained within the electronic game.

### DEFINITIONS

Agents—An agent is a character within an electronic game which behaves in accordance with programmed algorithms.

Avatar—An avatar is a digital representation within an electronic game whose actions reflect at least in part activities and/or instructions transmitted by the individual.

Game—In the context of the present invention, a form of activity or amusement utilizing one or more electronic devices that employ computer programming.

Health status—In the context of the present invention, the state of well being, including physical and mental well being, of an individual. This may refer to the general well being of the individual or the state of one or more measurable physiological attributes, e.g. hydration levels, body fat percentage, energy expenditure, motion, heart rate, etc.

Individual—A mammalian user of one or more devices and systems of the present invention.

Electronic Media—In the context of the present invention, media refers to the use of electronic games, interactive computer programs or other forms of electronically derived entertainment or lifestyle instruction, and includes the electronic elements supporting and/or displaying content.

### BRIEF DESCRIPTION OF THE DRAWINGS

FIG. 1—General example of the present invention.

FIG. 2—Illustration of one embodiment of the monitor of the present invention.

FIG. 3—Block diagram of one embodiment of health status determination by evaluator.

FIG. 4—Example of general steps of one embodiment for enabling evaluator response based on health status determination.

FIG. 5—Example of one embodiment of steps for enabling evaluator response based on health status determination.

### DETAILED DESCRIPTION OF THE INVENTION

The present invention claims the novel use of physiological monitoring combined with the automatic determination of one or more physiological parameters which then enables the delivery of a response utilizing this analysis to one or more forms of electronic media. In preferred forms of the invention such response is intended to result in a change in the health behavior of the individual.

To accomplish the method of the invention, the system of the present invention has at least one monitor having at least one sensor for the measurement of one or more physiological parameters from which one or more assessments of physiology and/or health status may be derived. Such assessments may include determination of calorie intake, energy expenditure, activity, hydration status, sleep patterns, stress levels, etc. In turn, such physiological parameter data may be conveyed to at least one evaluator substantially located within at least one electronic device. The evaluator may then generate a response based at least in part upon monitor data wherein the response may, in preferred embodiments, reward or serve to motivate or guide the individual's behavior towards improved health. This response is then conveyed to the individual through at least one form of electronic media. In preferred embodiments, the electronic media is in the form of a computer game having at least one avatar.

In general, the evaluator assessments and responses based upon physiological measurements are utilized to drive, at least in part, the electronic media's activities and user interactions, including the activities of agents, avatar roles and/or features, rewards, level of play, etc. within the media.

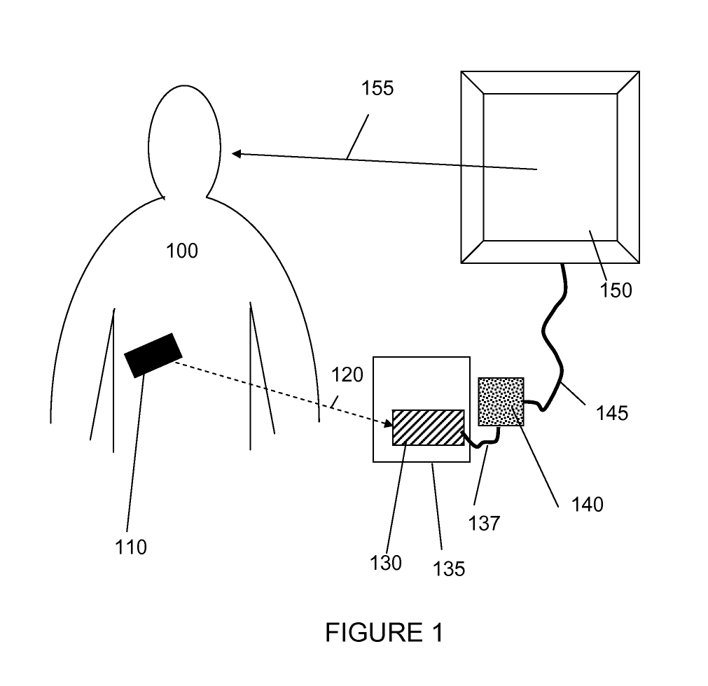

An example of a preferred embodiment of the present invention is outlined in FIG. 1. As shown, monitor 110 may be positioned on individual 100 and may measure one or more physiological parameters, preferably parameters related to health status over a period of time while the individual is not directly engaged with electronic media 140. The monitor may then transmit said data as shown by dashed arrow line 120 to evaluator 130 contained within electronic device 135 upon activation of electronic device 135. It should be noted that, in certain embodiments, evaluator 130 and electronic device 135 may be substantially contained within monitor 110 or alternatively, electronic device 135 and evaluator 130 may be substantially contained within electronic media 140. Evaluator 130 may then analyze measured physiological data and may determine a response to said analysis. This response may then be adjusted to the form of electronic media 140 and then conveyed to electronic media 140, thereby enabling adjustments to said media. The adjusted media may then presented to the individual, e.g. through video/audio display 150, as shown by arrowed line 155.

In various embodiments, a resultant behavioral change may then result in subsequent change in one or more measured physiological parameters, thereby enabling still further changes to said media in response to these physiological changes, thereby possibly offering a continued process of improvement in the health of the individual.

The system is described in greater detail below.

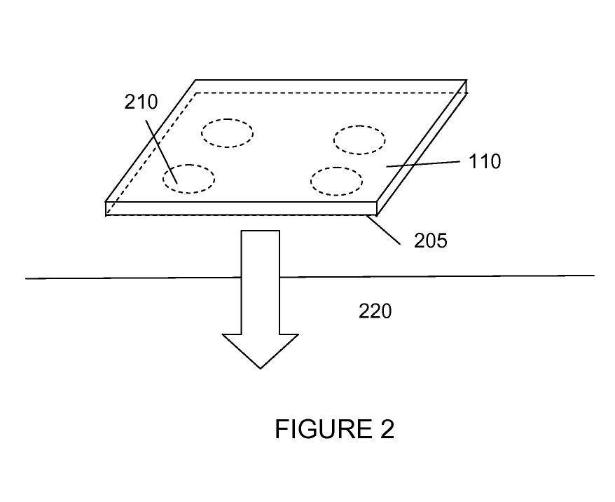

Monitor A representation of the monitor in preferred embodiments of the present invention is shown in FIG. 2. As shown, monitor 110 is effectively planar in form with a first surface 205 provide substantial contact of sensor 210 to body surface 220. In such preferred embodiments, the monitor is flexible and able to conform to body surfaces for user comfort and ease of use. In certain forms of these embodiments, the monitor may be affixed to the body, e.g. through the use of adhesives or straps. As indicated, the monitor of the present invention has at least one physiological parameter sensor.

In a preferred embodiment, sensors enable the determination of one or more physiological parameters related to metabolic status, e.g. sensors for body composition or energy expenditure. Such sensors may also enable the determination of calorie intake by the evaluator. That is, in certain embodiments, change in body composition, may be employed in the determination of energy balance resultant from the difference between the amount of energy consumed, i.e. calorie intake, and the amount of energy expended, e.g. through activity and basal metabolism. By knowledge of energy expended, the amount of calorie intake may be calculated. Such methods and devices are disclosed in U.S. patent application Ser. No. 12/386,614 “METABOLIC ENERGY MONITORING SYSTEM” which is incorporated in its entirety by this mention herein.

In general terms, sensors for assessing body composition may involve the exchange of one or more energies with one or more body regions in order to enable the assessment of composition or change in composition. Such sensors may include sensors utilizing electromagnetic, electrical, optical, mechanical or acoustic energies and the scope of the present invention is not limited to any one form or type of sensor. In a preferred form of the present invention, such sensors include bioelectric impedance sensors to enable determination of body composition in one or more body regions.

In preferred embodiments of this form of the invention, the body site to be measured for body composition or change in body composition is a body region preferably enabling assessment of a change in body fat. Examples of such regions may include the lower chest, abdomen or other body regions having significant storage of body fat responsive to changes in metabolic status. In such embodiments, sensor measurements may be substantially non-invasive, e.g. through the use of electrode-type sensors such as is the case of bioelectric impedance measurements utilizing electrodes in contact with the skin for the determination of body composition. In other embodiments, sensors for determining body composition or metabolic status may be invasive, e.g. implanted and in direct contact with one or more selected body regions or tissues.

In still other forms of the invention, other forms of energies and energy exchange may also be utilized for the determination of body composition. Examples of such forms of measurement may include the use of ultra wideband radar or near infrared optical measurements. In certain instances, forms of sensors may take advantage of one or more energies radiated from one or more body regions, e.g. thermal mapping, in order to determine underlying body composition. In yet other embodiments, sensors for one or more analytes, e.g. circulating hormones such as leptin or metabolites reflecting changes in body composition such as exhaled ketones or carbon dioxide, may be utilized either alone or in conjunction with additional sensors for the determination of body composition changes.

In still yet other embodiments, multiple sensors inspecting one or more body regions having same or different forms of energies may be employed to enable determination of body composition, body composition changes and/or metabolic status. In related forms of the invention, sensors useful for the determination of body composition and body composition changes may also utilize data from other forms of body measures, e.g. weight scales, one or more body dimensions or specific gravity/buoyancy determinations, to aid in the overall analysis.

In addition to sensors for body composition determination, the method and devices of the preferred embodiment of the present invention may also employ one or more sensors for the determination of energy expenditure. In general terms, these sensors are responsive to activity or energy expenditure of the body. As such, these sensors may measure one or more body parameters associated with activity, e.g. heart rate, core temperature, or motion (e.g. through the use of one or more accelerometers), from which overall energy expenditure may be determined. In a preferred embodiment of the invention, sensors enabling the determination of heart rate, motion and core body temperature are utilized and are located on the same monitor utilized for the determination of body composition.

The sensors utilized for energy expenditure may also be the same as one or more sensors utilized for body composition determination. For example, sensors enabling ultra wideband radar measurements for body composition determination may also be employed for the determination of heart rate and/or respiration rate thereby providing data useful for energy expenditure determination. Measurements used for these respective analyses, i.e. body composition versus energy expenditure, may be the same or different. For example, regional ultra wideband radar data utilized for energy expenditure determination may employ time-based measurements, such as to enable heart rate and/or respiration determination, whereas effectively instantaneous point in time measurements may be utilized for determination of the composition of the underlying tissue and therefore useful for body composition determination. However, other sensors may be employed for the determination of energy expenditure and the scope of the present invention is not limited to one form or type of sensor.

As noted above, a preferred embodiment of a monitor of the present invention is that of a planar conformable structure such as shown in FIG. 2. In such forms, sensors enabling the determination of body composition and/or body composition change as well as of activity may be incorporated into the monitor. In such embodiments, one or more means of transferring information to an evaluator and/or display with electronic media may be employed such that the monitor may remain continually affixed to the individual, e.g. through adhesives, for an extended period of time, e.g. hours or days.

In an alternate embodiment of the present invention, one or more sensors, e.g. acoustic, electrodes, accelerometers, or ultra wideband radar, are connected to or contained within a cellular telephone or a cellular telephone-like device having cellular phone communication functionalities and a display. In such embodiments, the monitoring function may entail periodic use by the user, e.g. placement of a sensor against a body region, during a game activity and/or while not engaged in a game activity. In addition, other sensors, e.g. motion sensors such as accelerometers and/or movement/distance sensors, e.g. GPS, may be activated automatically. Such automatic activation may enable determination of user status such as position, activity, etc. and thereby facilitate determination of additional physiological parameters, e.g. energy expenditure. In such embodiments, the monitor, evaluator and media/display functions may be effectively contained within a single unit, e.g. a modified cell phone. In addition, such embodiments have additional methods of enabling user input, e.g. keyboard, as well as providing, in part or in whole, necessary electronic circuitry, memory and power to enable the present invention.

In form, the sensors for body composition and/or energy expenditure determination may be configured in a variety of fashions and the scope of the present invention is not limited to any one form of sensor.

In yet other embodiments, other sensors may be utilized to determine other health parameters. Such sensors may include sensors enabling the determination of hydration status. Such sensors and monitors are disclosed in U.S. Pat. No. 7,783,344 “HYDRATION MONITORING” which is incorporated in its entirety by this mention herein. Sensors/parameters may also include range of motion sensors useful for assessing rehabilitation of a body part, e.g. leg, following an injury or surgery, cognitive awareness sensors, e.g. sensors enabling the recording change in brain wave patterns or heart rate variability reflective of stress/relaxation, sensors for the determination of blood oxygenation levels associated with respiratory function or sensors evaluating socialization and the degree/extent of interaction with others, e.g. sensors recording voice activity or voice stress levels.

In various embodiments, sensors may be in direct contact with the body or body surface. Examples of such sensors may be surface electrodes useful for the determination of body composition or heart rate. In other embodiments, sensors may be located in monitor structure but need not be in direct contact with the skin surface. Examples of such sensors include ultra wideband radar or accelerometers to enabling the determination of respiration rates and/or motion relating to energy expenditure or temperature sensors enabling the determination of ambient temperatures. In yet other embodiments, sensors may consist of sensors enabling the determination of one or more physiological parameters through the use of sampled biomaterials, e.g. saliva, tears, blood, or exhaled breath. Such embodiments may also include sensors responsive to ingestion of food or calories, e.g. sensors responsive to glucose levels or other circulating metabolites. Such sensors may include sensors able to respond to detect changes in dermal thicknesses associated with glucose levels. Sensors may be continually borne or used in a periodic or intermittent fashion.

In short, sensors and monitors may be utilized to measure one or more physiological parameters and configured in a variety of ways, including configurations that may be implanted within the body, affixed directly to the skin surface, handheld by the individual, incorporated into articles of clothing, or be affixed to furniture, bedding, or walls. Numerous examples of possible sensors are conceivable and the scope of the present invention is not constrained to any one form or type of sensor.

Numerous embodiments of monitors as well as of sensors are conceivable. Such monitor embodiments include monitors in the form of devices for placement in or about the ear, watch-like devices to be worn on the wrist, incorporation into cellular phones, into handheld game controllers, or monitors with remote sensors affixed to walls or structures. As an example of one alternative embodiment for the monitor, the monitor with sensors may be incorporated into a handheld device, e.g. into a suitably configured cellular phone. In such form, sensors such as ultra wideband radar may be enabled and utilized by periodic placement of the sensors at selected body sites, e.g. against the lower chest, to provide data suitable for determination of one or more physiological parameters, e.g. body composition. In addition, incorporation of additional sensors such as sensors for activity, e.g. accelerometers, may also be included within the body of the device, to enable motion detection or the sensing of other physiological parameters not requiring direct contact with the user.

In yet other embodiments, multiple monitors are employed, e.g. one monitor may be employed that enables measurement of one physiological parameter, e.g. body composition, and is physically distinct from a second monitor employed for measuring a second physiological parameter, e.g. energy expenditure. Examples of such multiple platforms include use of pedometers or other activity sensors combined with one or more patches affixed to the body enabling measurements of body composition.

In addition to sensors, the monitor preferably contains electronic circuitry necessary for the function of the sensor, e.g. memory, digital signal processor, analog to digital converter, digital to analog converter, amplifiers and power (battery), as well as a means of communicating sensor data to one or more evaluators for analysis and subsequent display. The method for constructing such devices is well known to those skilled in the art of physiological monitor design and construction.

In general terms, communication from a monitor may be two way, wireless or wired, e.g. through radio transmission or by direct connection. In certain instances, the circuitry utilized for one or more sensors, e.g. ultra wideband radar, may be utilized at least in part for communication. In preferred embodiments, such communication utilizes wireless, e.g. radio wave-based, forms of communication, thereby facilitating monitor use while the individual is ambulatory.

In addition to the communication of data to one or more evaluators, such communication may also include instructions or directions transmitted to the monitor from the evaluator, e.g. for additional measurements, different forms of measurements or for status of the monitoring platform such as battery life. In yet other forms of communication, one or more identifiers associated with the monitor and/or sensors may be transmitted to the evaluator. Such conveyance of identifiers may facilitate identification of individual components of the monitor to aid in assessment of performance, for assisting in the identification of an individual's data set within a group of individuals utilizing a common evaluator, e.g. within a localized multiplayer gaming scenario. Alternatively, identifiers may enable the automatic coordination of data from multiple users at remote locations, e.g. as in the case of multiplayer games utilizing communication employing the Internet or to aid remote individuals involved with game activities, e.g. reward providers, in the verification of an individual for reward receipt.

One or more monitors and/or sensors may be used for short periods of time, intermittently, periodically, for extended periods of time, e.g. several hours or days, or effectively continuously. In preferred embodiments, effectively continuous measurements of a single body region are employed thereby facilitating detection of change in one or more measured parameters in the monitored body region.

One or more different types or forms of monitors and/or sensors may be employed for the purpose of monitoring. For example, an individual may utilize a form of monitoring enabling determination of sleep patterns, e.g. monitoring brain waves and/or activity, heart rate and respiration rate combined with time of measurement, while not directly engaged with the electronic media. Then, when engaged with the electronic media, the individual may utilize a monitor suitable for determination of hydration status and energy expenditure. Such a scenario is conceivable when utilizing electronic media is intended to lessen stress where evaluation of sleep patterns with physical fitness through exercise are combined to assess stress levels and possibly enable improvement of stress levels through one or more exercise activities.

In a preferred embodiment, the individual employs one or more monitors and/or sensors while not directly engaged with the evaluator/media components. For those periods of time when the individual is not directly engaged with the evaluator/media, the physiological data may be stored within the monitor then automatically transferred to the evaluator/media components upon activation of the evaluator/media components. This transference enables the physiological data history of the individual through this intervening period of time to be automatically compiled and utilized by the evaluator.

Monitoring may include the use of time and/or date information included into the data set such that the period of time and/or of duration of a monitored physiological parameter and data values may then subsequently be analyzed by the evaluator. Such inclusion may enable the data be subsequently analyzed in a variety of ways, e.g. for a length of time a physiological parameter is greater than or less than a predetermined value, or for the coordination with one or more data sets from other data sets. For example, a monitor plus sensors worn in effectively continuous fashion may enable the subsequent determination of sleep patterns, e.g. duration, intensity, by the evaluator.

In addition, use of multiple monitoring measurements and employment of time/date information facilitates the analysis of trends in physiological data overtime as compared to a single summary of data corresponding to the period of time when the individual is not engaged with the media. For example, a monitor for the purpose of determining energy expenditure, e.g. a heart rate sensor plus accelerometer sensor, effectively continuously affixed having timing capabilities may enable the subsequent determination of peak energy expenditure during exercise or activity. This contrasts with a monitor without such temporal capabilities which then may record a cumulative amount of energy expended without regarded for maximal times, peak levels, etc.

Additional data may be included in data supplied to the evaluator where such data is generated by one or more additional sensors or methods not directly associated with the devices of the present invention. For example, the individual may periodically weigh themselves and supply this information to the evaluator in order to track their progress towards a desired goal. As a second example, an individual may compile a diary of foods consumed and activities performed and provide this information to the evaluator such that additional data may be available to the evaluator for determining health status and adherence to suggested lifestyles or dietary regimens.

Electronic Device A primary function of the electronic device of the present invention is to contain and enable the function the evaluator of the present invention. In order to accomplish this enablement of evaluator functions, the electronic device may consist of electronic circuitry, power, housing, control buttons, switches, displays, etc. Such elements enable the receipt of monitor data, the storage and processing of such data by the evaluator and the conveyance of evaluator analyses to one or more electronic media. In various embodiments, electronic devices of the present invention may also have additional input devices, e.g. keyboards, touch screens, voice recognition capabilities, etc., such that other data, e.g. non-monitor data, may be supplied to the evaluator.

In form, the electronic device may be an independent device, or all or a portion may be contained within a monitor and/or electronic media (and a device supporting the electronic media). Multiple variations of electronic devices are readily conceivable and the scope of the present invention is not constrained to any one type, form or style of electronic device and such devices are readily constructed by those skilled in the arts of electronics.

In various embodiments of the invention, communication, e.g. the transmission of one or more data sets between monitor and the electronic device, or between electronic device and electronic media (and the device supporting the electronic media) may be done either through wired or wireless fashion. In select embodiments, said communication is encrypted to ensure that the data remains restricted to the individual. The scope of the present invention is not limited to any one form or type of monitor and/or mode of communication.

Evaluator In preferred embodiments of the present invention, the evaluator may be utilized to analyze one or more sets of physiological data received from one or more monitors. In preferred embodiments, this analysis may take the form of comparison of measured data to one or more predefined physiological (or health) status goals, objectives or metrics. Multiple forms of comparisons are conceivable. For example, such comparisons may include comparisons made using simple values or reference points, e.g. total energy expended or percentage body fat lost over a period of time, or may entail more complex analyses and comparisons, e.g. utilizing data from multiple sensors and/or incorporating factors such as trends or patterns over time. In still other embodiments, portions of the monitored data may be employed by the evaluator, e.g. the determination of periods of time and/or extent to which a monitored physiological parameter exceeded a predetermined level. Accordingly, the scope of the present invention is not constrained to anyone form of comparison and/or analysis process.

In various embodiments of the present invention, objectives, goals or metrics to be utilized for the purpose of comparison may be established by the individual or provided automatically, e.g. by the evaluator as part of a predetermined functionality of the evaluator. Such predetermined functionality may also reflect the form and type of electronic media to be utilized and the intended audience for such media, e.g. electronic games with role playing avatars and agents or health/fitness programs.

In preferred embodiments, upon determination of the relationship between one or more measured physiological parameters and desired goals, objectives or metrics, the evaluator may provide a forward looking response, e.g. to promote desired behavior or activity by the individual during future periods of time. Alternatively, the evaluator may provide a retrospective response, e.g. as a reward for one or more determined changes in one or more physiological parameters.

In addition, the evaluator in preferred embodiments may provide at least one response based upon said analysis that is conveyed to one or more electronic media for subsequent conveyance to the individual.

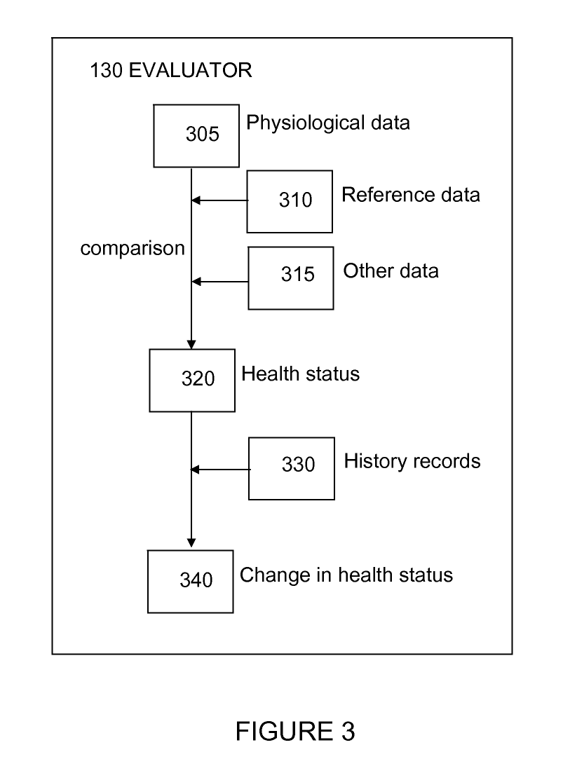

As noted above, a preferred function of the evaluator is the comparison of measured physiological data to one or more predefined physiological status goals, objectives or metrics. An illustration of one form of this embodiment is shown in FIG. 3. As shown, received monitor data 305 is compared to one or more reference data tables 310 by evaluator 130. Reference data tables may consist of physiological parameter data correlated to various degrees of health status associated with this measured parameter. To assist in the determination of health status, additional data 315, e.g. age, gender, etc., may be utilized. This data may be requested by the evaluator as needed or may be already present within the evaluator, such as individual data entered previously during initial system set-up. In other embodiments, reference data tables 310 may be updated or renewed through outside communication from one or more remote data systems to improve the accuracy of the physiological parameter and/or health status assessment. In certain instances, the health status assessment may be identical to the status of the measured physiological parameter.

In other embodiments, formulas or algorithms enabling assessment of physiological parameter and/or health status may be employed in place of or in supplement to reference data table 310. For example specific algorithms may utilize physiological parameter measurements such as bioelectric impedance data to calculate body fat percentages. Additional data inputted by the individual, e.g. age, weight, height, gender, may be utilized to enable algorithm calculations.

In these and other embodiments, the value of or change in value of one or more measured physiological parameters, e.g. body fat percentage loss, relative to a desired state may be determined. In such embodiments, desired health values and or desired physiological parameters, e.g. body fat percentage, may be directly inputted into the evaluator, e.g. by manual input through a keyboard by the individual. In alternate embodiments, the desired physiological parameters may be automatically determined by the evaluator, e.g. through the use of one or more tables and/or algorithms enabling tailoring of a desired physiological parameter value to the individual. In such embodiments, additional data may be inputted by the individual, e.g. age, gender, weight, height, etc., to enable the algorithm calculations. The evaluator may then utilize one or more measured physiological parameter data to determine the difference between the target physiological parameter value and the measured physiological parameter value(s).

Upon determination of the health status of the individual 320, the evaluator may also determine a possible change in the health status 340 as compared to a prior health status. As part of this assessment, the evaluator may also employ one or more recorded data sets, i.e. history records 330, to enable determination of change, the magnitude of change or the trend of change in one or more physiological parameters and/or health status over a period of time. In alternate embodiments, such history data may include data sets transmitted from the monitor, e.g. physiological data periodically recorded over the period of time while the individual was not in contact with the evaluator or when the evaluator was not activated.

In general terms, change in health status assessment (or health status in absence of prior data) 340 may comprise one or more of the following findings: no change, positive change or negative change relative to a prior health status assessment, dependent on the parameter measured and the assessment made. In certain embodiments of the invention, these findings may be quantified to enable gradations of response to be made. In those instances where there is no prior health status record to reference, e.g. as in the initial use of the system, the evaluator may make a health status assessment of no change to enable a starting point to be constructed. Alternative embodiments for evaluator function for assessment of health status and change in health status are conceivable and the scope of the invention is not limited to the example presented herein.

For example, consider the scenario wherein the individual over a period of time, e.g. the interval between usages of the electronic media, has lost substantial body fat. The monitor, through use of one or more sensors, may measure one or more parameters associated with body fat and convey these data to the evaluator. The evaluator in turn, by comparison to previously acquired body composition physiological data, e.g. the initial body fat data, then may determine the extent of change in one or more of these data. The value from this comparison may then be compared to desired body fat change objectives contained within the evaluator to provide a response metric, e.g. a numeric value reflective whether said chance met, exceeded or fell short of the desire physiological parameter and/or health status goal.

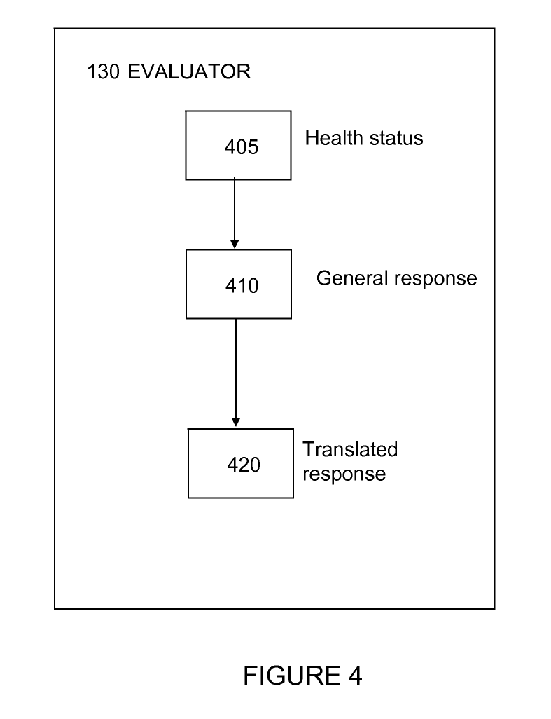

It follows then that a second general function of the evaluator is to create a response intended to be conveyed to the individual through the context of the electronic media based upon the evaluator assessment of health status and/or that of one or more physiological parameters. In this context, health status or physiological parameter may refer to the individual's current health status or physiological parameter, change in health status or physiological parameter relative to a prior state or a combination of the two. In preferred embodiments, this response is adapted to the context of the media environment employed, e.g. the game context. FIG. 4 illustrates one example of this process. As shown, this example is comprised of two general stages. The first stage is the determination of a general response 410 by evaluator 130 based upon health status change 340. The second stage is the translation of response 410 to one appropriate the context of the media employed 420. As such, this second stage response may differ between applications as needed to better fit different applications and electronic media formats/contents.

In general terms, a response may be forward looking, e.g. responses intended as motivations or inducements towards change in one or more physiological parameters to be assessed in future periods of time, or retrospective, e.g. as a reward for measured changes in one or more physiological parameters that have occurred during a prior period of time, or a response may be a combination of both forward looking and retrospective actions.

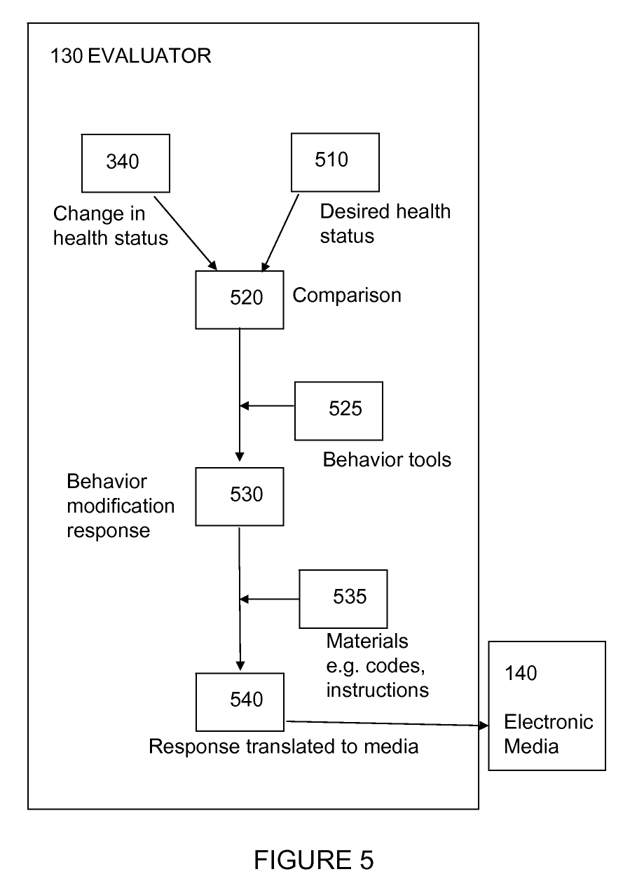

An example of how an evaluator may construct a forward looking embodiment of a response is illustrated in FIG. 5. As shown, a first activity by evaluator 130 is the comparison 520 of measured change in health status 340 (or a current health status—not shown) to desired health status 510. Desired health status 510 may have been previously inputted by the individual, or inputted by a third party, e.g. clinician. Alternatively, desired health status 510 may be determined by the evaluator through the use of appropriate population tables and health forecast statistics and/or predictive algorithms of health status.

Once comparison 520 between current and desired health status has been made, e.g. a numerical value such as a percentage indicating the degree of difference between the current and desired values, the evaluator then determines an appropriate forward looking response or responses. For example, evaluator 500 may utilize one or more sets of established psychological behavioral modification tools or processes 525 to derive response 530 that is intended to result in behavior that will lead to a decrease in comparison 520, i.e. the difference between the desired versus current health status. In certain embodiments, difference 520 may be of such magnitude that only a portion of this difference is sought to be resolved through incremental changes or goals, i.e. an incremental approach may be adopted in order to maintain individual participation and useful behavior.

Tools or processes 525 may include motivational structures that: afford the individual behavioral choices from which they may select; provide the individual with objectives that will have high likelihood of success and therefore increase individual self confidence and self worth; utilize other individuals or agents to form a support network for the individual; and employ incentives that may encourage desired behavior. Forms of such tools and/or processes are well known to those skilled in the art of behavioral intervention. In general terms, the present invention may utilize one or more behavioral or motivational tools to encourage desired behavior, including but not limited to: educational information; expert advice; support networks based upon other individuals or game players; or incentives. As one may readily conceive, aspects of each of these tools may overlap with aspects of one or more other tools in practice.

In various embodiments, educational information may be information regarding lifestyle changes to be adopted, diets to be selected, recommended exercise patterns, the impact of various environmental factors, e.g. calorie content of foods, impact of short sleep duration on alertness, etc., on health or information regarding the individual's own health status and progress towards desired health objectives. Multiple forms of education are possible and the scope of the invention is not limited to any one form of education.

Likewise, expert advice may be comprised of many different forms within the scope of the invention. Illustrative examples include the presentation of stored input from experts in the particular health area to be improved, e.g. weight management, or the direct interaction between the individual and one or more experts being mediated through the game environment. In certain instances, the expert advice may be contained within the evaluator. In alternate embodiments, the expert advice may be provided by a remote service using either live or preprogrammed interactions to better meet the needs of the individual.

Support networks for enabling individuals to meet health objectives may be comprised in a variety of fashions. In one instance, these may be through the establishment of groups of individuals who are players within an on-line game and thereby support each other through the context of the game objectives. In alternate embodiments, the support network may be through other individuals pursuing similar objectives and thereby coordinated in their communication by intercommunication between the evaluators of these individuals. In yet other forms, the support network may be comprised of one or more individuals involved in other social structures, e.g. Internet social media sites, and/or outside support systems such as employed by weight management firms. In such embodiments, one or more results of monitored parameters and/or evaluator assessments may be provided to these groups to facilitate support for the individual. Multiple forms of support networks and modes of communication between members of the network are conceivable within the scope of the present invention.

Another form of motivational tool is that of incentives. Incentives may take the form of tangible or intangible items. Examples of tangible items include the use of cash awards if certain milestones towards a desired health status are met by the individual or the acquiring a gifts or gift cards, e.g. a reward card from electronic game manufacturer, based upon progress to a milestone.

Examples of intangible rewards include the use of points awarded within the game context or ability to obtain one or more changes to the features of the electronic media, e.g. game, through the individual's progress. In various forms, points or other symbolic representations of progress towards a health status goal, e.g. virtual currency, may be utilized to support, guide or otherwise motivate a user of the present invention. For example, accumulating points over time may result in the ability of the user to progress to a more advanced stage within the context of the electronic media. The potential of acquiring these points may serve as an inducement to the individual to alter their lifestyle which in turn would lead to a change in a monitored parameter and thereby enable the future awarding of these points. Points may be shown as a score within the electronic media and/or enable the individual to advance to a different level of the media, e.g. game level. In other embodiments, the accumulation of points may be translated into tangible rewards such as the award of a gift card, etc. In yet other embodiments, where the individual is involved with a group, the points may contribute to the overall point total of the group. Such overall group point totals may be in a variety of fashions, e.g. virtual currency may be used to achieve a tangible reward for the group or may be used as a competitive metric relative to one or more other participating groups of individuals.

In certain instances, intangible rewards may include negative rewards, e.g. the loss of points, virtual currency or features of one or more agents or avatars, based upon the individual's progress.

In preferred embodiments, one or more of the above tools for motivating the individual to change behavior in a desired fashion is incorporated into the response of the evaluator.

Other forms of responses may those considered as retrospective in nature, e.g. serving as rewards for achieving one or more desired physiological metrics. For example, intangible or tangible rewards such as those described as incentives above may serve as forms of responses based upon the meeting or not meeting of one or more goals or health objectives. That is, upon the measured change in one or more parameters consistent with achieving a desired change in a physiological parameter, e.g. fat loss of a specified percentage, then the individual may then be rewarded through the response.

Upon determination of a response 530, either forward looking or retrospective, the response 530 may then be translated into a form 540 more appropriate to the media being utilized such that the behavioral change integrated within the context of the media. This translation may be accomplished by use of materials 535, e.g. codes or instruction sets within the electronic media software accessible by the evaluator enabling change to a portion of the media content or format. Upon this translation, response 530 is now suited for incorporation into electronic media 140 and conveyance to the individual. In other embodiments of the present invention, response 530 may be conveyed to electronic media 140 without prior translation. In such instances, electronic media 140 may perform any necessary translations of response 530 prior to conveying the response to the individual.

In preferred embodiments, response 530 is converted 540 and transferred to electronic media 140 automatically. In other embodiments, response 530 is converted 540 then upon command transferred to media 140. In still other embodiments, response 530 is stored for future use, conversion 540 and possible transference to media 140.

In preferred forms of the present invention, such translation into the context of the electronic media is made in a substantially proportional fashion. For example, if the measured physiological parameter is body fat change, the proportion of this change relative to a target value may be determined by an evaluator. The proportion may then in turn be translated into numeric values, either positive or negative, for use in the electronic media, e.g. as points awarded to the individual or representative of the individual (avatar).

In alternate embodiments, the translation may take the form of adapting a non-numeric response, e.g. behavioral advice, to the electronic media. In such embodiments, use of set translation tables, e.g. conversion relationships between general responses to specific electronic media responses, may be employed and/or rules engines deriving such conversions from general to media specific values may be utilized.

By way example of how the evaluator may accomplish a translation to media, consider a scenario wherein the evaluator has determined that a health status change, e.g. to lose body fat, is needed to meet a previously entered health goal. The evaluator may determine a form of response, e.g. game points to be awarded if body fat is lost, and a behavior motivational approach tailored to the individual, based on profiles for typical users of the game as well as characteristics of the individual. The typical user profile may be accomplished using a rules engine having approaches found to be useful to change one or more behaviors for these individuals that results in the desired behavior. The individual motivation may be accomplished by evaluation of the individual's prior motivational history to determine those tools found to be successful or unsuccessful. Alternatively, such rules engines may employ data sets obtained from larger populations of tools found to be successful for achieving the same or similar objectives.

A motivational approach such as exercising vigorously before dinner while avoiding snacks may be suitable as a motivational tool/guidance for adolescent males however a walk during lunch with fruit for lunch may be more suited to an older demographic. Rules engines may incorporate such information to be utilized in formulation of the general response which is then converted to the context of the electronic media. In the context of the above example such conversion may include an agent within a game scenario visibly demonstrate the desired actions to the adolescent male whereas the recommendation for a walk during lunch plus fruit may be provided as a textual message within a health/wellness interactive program. Accordingly, a rules engine responses may be tailored to the individual using the media as well as the media content itself.

Electronic Media In the present invention, electronic media may be utilized to convey one or more evaluator responses to the individual. Adjustment of the visual and/or audio output of the electronic media represents a preferred method of communication or conveying these responses. However, alternate forms of communication, e.g. haptic feedback through game controllers or interactive text messages conveyed to cell phones, are readily conceivable. In yet other embodiments, the responses may be distributed to more than one forms of media, e.g. more than one game, or to more than one media site, e.g. to multiple individuals located at different locations simultaneously. Accordingly, the scope of the present invention is not limited any one form of communication or conveying of responses through electronic media.

As part of conveying the response to the individual, the evaluator response, in preferred embodiments, may be adapted to the context of the electronic media, e.g. the game. In certain embodiments, the media itself may be able to accept inputs, instructions or commands enabling the response to be conveyed in the context of the media. In preferred embodiments, this adaptability is in the form of set of commands triggered by one or more evaluator response inputs already tailored to the media content, e.g. avatar commands. In alternate embodiments, a standard or universal set of health response inputs may be translated by the electronic media to fit to the specific needs of the media content. In such instances, the evaluator may not create a response tailored to the electronic media.

In order to accept and utilize one or more responses from an evaluator, the electronic media preferably incorporates one or structures and processing software enabling the reception and incorporation of one or more responses. For example, means to receive and process a response may include wired or wireless communication capabilities, e.g. antenna plus necessary electronics and communication software. Alternatively, the evaluator may be incorporated within the electronic media and therefore the electronic media not require significant reception and/or incorporation structures. However, in such instances, the electronic media/evaluator may require ability to receive data from monitor and therefore still may possess some form of communication capabilities.

Once a response has been received by the electronic media, the media may be adjusted to incorporate this response. To accomplish such adjustments, in preferred embodiments, the electronic media software is constructed to accept a predetermined range of responses, e.g. numerical values, previously adapted to the electronic media format and content, and then as appropriate incorporates these into the media. For example, upon receiving a response indicating that a user has met one or more objectives and accordingly received a number of game points as a reward, these game points may be provided or shown on a display screen to the individual such that the individual is now aware that the points are awarded and may be utilized within the content of the media, e.g. to provide more functionalities to an avatar or to enable advancement to a higher level of play or interaction.

Forms of adjustment by the electronic media to convey the response of the evaluator may include the direct presentation of information to the user, e.g. the presentation of one or more incentives, points or support messages. Alternatively, the conveyance of information may take the form of a change in media, e.g. a change in the role or in the capabilities of an avatar representing the individual within the media, or a change in the electronic environment surrounding the avatar. Such changes may include the addition/removal of avatar functionalities, the addition/removal of features such as tools, weapons, devices available to the avatar, the addition/removal of agents or types of agents interacting with the avatar, or change in the electronic environment such as shifting to a different play level either more or less advanced in complexity/difficulty.

In addition to conveying a response to an avatar, the response may be conveyed through one or more agents interacting with the avatar and thereby conveyed to the individual. Such agents may take the form of adversaries, advisors or companions within the context of the game. In such embodiments, the response may be provided verbally or through actions of the agent. In addition, the response may take the form of messages, symbols, numerical values or other forms of response, e.g. visual, audible, or tactile.

In yet other embodiments, one or more animated or still figures representing the individual and/or the health status of the individual may be conveyed by electronic media. In such instances, responses may indicate the present status of the individual, the past status of the individual or possible future statuses of the individual if health status is modified or not. Such conveyances may also include animations of anticipated lifestyles and/or physical attributes of the individual.

In general terms, the ability to enable adjustment of electronic media to accommodate one or more responses provided by one or evaluator are well known to those skilled in electronic media arts, e.g. gaming software, and the scope of the present invention is not constrained to any one form or type of adjustment.

A variety of forms of interaction between the individual and with the electronic games or instructional materials are conceivable within the scope of the invention. Examples of these interactions include games where the individual directly interacts with the game through the use of a controller or other type of sensor, e.g. motion wand. In such games, the individual may be represented as an avatar within the context of the game or instructional material and directly interacts with the media.

By way of example, consider the following scenario wherein the general behavior response is for the individual to reduce their calorie intake relative to their energy expenditure by ˜300 kcal/day for 7 days (i.e. lose ˜0.5 kg of weight). Accordingly, some form of behavioral change is required on the part of the individual. A general response formulated by the evaluator may be to consume fewer calories and to increase exercise levels.

In one form of a tailored response, this general response may be translated into a form of an agent suggesting that the individual take a walk each day during lunch and leave out at least one desert or snack per day. This form may be appropriate for the content of the electronic media such as an interactive health program. As support and incentive, the interactive health program agent may lead to a combination of messages, e.g. a congratulatory message and a note alerting to possible points towards a reward, e.g. a coupon for a reduced rate at a day spa, if successful completion of a goal is met.

In contrast, in the context of an interactive action computer game where the individual may role play as an avatar within the game, the response may be presented differently. That is, agents within the game may suggest to the avatar that additional weapons or capabilities would be forthcoming if the avatar became more fit—by limiting consumption of food snacks and getting increased exercise during periods of time between game sessions. In both scenarios, the general response, i.e. response 530, is consistent—reduce calorie intake relative to energy expenditure, resulting in weight reduction, however the method by which this accomplished is tailored, i.e. conversion 540, to the intended electronic media (game/video) context and through this conversion conveyed to the individual.

In preferred embodiments, the individual interacts singly with the electronic media, thereby receiving responses from the evaluator tailored solely to this individual. In alternate embodiments, two or more individuals may simultaneously interact either with the electronic media or with each other through the media and thereby may share evaluator responses. In still other forms, the individual (or groups of individuals) may interact with one or more remote individuals who share the electronic media content and receive group evaluator responses to their health status as a group.

In yet other forms, one or more third parties may be apprised of the individual and their progress within the game and respond to this progress within the context of the game environment. These third parties may choose to offer additional incentives, advice, guidance or support to the individual. Such third party activities may also take the form of advertisements, solicitations or offers generally related to the individual and/or the individual's health status. In certain embodiments, the individual may be afforded the opportunity to purchase or obtain an additional program or materials in support of their goals and/or as a supplement to media, e.g. game, activities.

Additional forms of interaction with the electronic media are conceivable and are within the scope of the present invention.

### EXAMPLES OF USE

Representative examples of various forms of the present invention are presented below.

### Example 1

### Role Playing Game Incorporating Weight Management

In this example, the electronic media is one of an action game wherein one or more individuals are avatars within the game, e.g. a medieval quest with agents such as wizards, warriors, dragons, and other characters. Accordingly, the individual may be an adolescent whose primary purpose for participating in the game is not for the purpose of health improvement but for the purpose of competition against the game.

In use, the individual may affix a monitor, e.g. a patch, onto their body at a specified location, e.g. the lower chest. Such initiating activity may also serve to automatically activate the monitor, e.g. by opening the package containing the monitor. In this example, the monitor may be one intended to measure physiological parameters associated with body composition and activity. The user may also activate, e.g. turn on using a switch, the evaluator and the electronic media (an electronic game). The monitor that is affixed to the individual may automatically communicate in a wireless fashion with the evaluator. The evaluator may then employ the electronic media to verify through an interactive display that the individual is wearing the monitor. In addition, the evaluator may ask for additional information regarding the individual, e.g. gender, age, height, approximate weight, such that a more accurate assessment of health status may be made. The individual may then engage with the electronic game through the use of a keyboard, video controller/joystick or other similar device. The avatar available to the individual may be set in part based upon evaluator determination of the individual's fitness relative to body composition. That is, various play levels within the game are available but these may be selected in part based upon evaluator assessment of health status, e.g. body composition—lean, normal, overweight or obese, for the individual's gender and age using standardized tables.

During the course of the interactive game, one or more agents within the electronic media may offer an evaluator response as support or advice as to how to improve the avatar's capabilities. For example, an agent may suggest to the individual through the individual's avatar that additional features or functionalities such as more spells or strength, may be gained by the avatar through improvement in lifestyle over the next few days. The agent may further suggest activities in the context of the game role that will modify behavior in healthy directions, e.g. desserts or sweets as draining vital energy and therefore should be avoided or the need to be in the sun and walk to acquire additional energies. The individual then may continue game play for a period of time then stop. The individual may continue to wear the monitor however.

During the intervening period of time before resuming game play, e.g. a few days, the individual may adopt some or all of the agent's suggested behavioral changes to improve their avatar performance. After this interval of time, the individual then resumes play. As one possible evaluator function, the evaluator automatically analyzes received physiological data to determine that the same individual and patch is communicating with the evaluator. In effect, the physiological data may comprise a signature with defined characteristics and limits of anticipated change over this time period that would preclude transference to other individuals or misuse of the data.

The evaluator may automatically assess changes in body composition as well as the activity levels during the intervening period of time between gaming sessions from which a change assessment of health status is made. This change assessment is then related to a response by further evaluator activities, e.g. reward for positive changes in body composition or increased activity. This response is then incorporated into the game such that the individual's avatar may receive a reward such as an increase of powers/functionalities to the avatar. Further levels of play/difficulty may then be offered for further improvement in avatar health. In effect, the individual may become healthier in order to improve avatar performance.

### Example 2

### Lifestyle Instruction Interactive Program

In this example, the individual may desire to improve their health through stress reduction. A typical individual therefore may be a middle aged adult actively trying to improve their health. In use, the individual may affix a monitor able to sense sleep patterns and stress, e.g. motion sensors (sleep) and heart rate sensors enabling determination of heart rate variability (stress).

The individual may wear the monitor for a period of time prior to commencing the interactive program to establish a baseline set of physiological parameters. Upon initiation of the program, the evaluator may obtain information regarding the individual through a query system employing the electronic media of the interactive program. Such information as age, gender, work hours, normal rest periods, etc., may be obtained. From these baseline parameters, the evaluator may make suggestions as to method to improve health or well being during sessions with the interactive program. These suggestions may include one or more agents demonstrating techniques for stress reduction, e.g. meditation techniques. In addition, the agent may suggest lifestyle changes, e.g. change of sleep duration, in response to measured physiological parameters. Success or failure of these suggestions will be subsequently registered by the evaluator over periods of time, including those periods when the individual is not actively engaged with the program but is still wearing the monitor.

### Example 3

### Elderly Assistance Program

In this example, the system of the invention is utilized to support behavior change to improve hydration, eating or exercise habits in elderly individuals. In form, the electronic media may be presented as a dedicated agent within a display system who functions as a companion to the individual and who provides interactive experiences with the individual. The display system also contains evaluator functionality. Such display systems may be dedicated units conveniently located in a household area that is conducive to behavioral modification. One such location may be the kitchen wherein the agent may be able to interact with the individual on a frequent basis.

In use, the individual may on a weekly basis affix a monitor, e.g. a patch, on their body in the indicated location, e.g. lower chest. The individual may then verbally or through a touch screen, alert the evaluator that the patch is on. In certain circumstances, the evaluator may automatically be alerted to the activation of the monitor which may occur upon placing the patch on the body. The agent may then verbally query the individual to affirm that the monitor is being worn. Throughout the course of the period of use, the monitor may provide physiological data relevant to the determination of hydration status and body composition as well as of sleeping patterns and activity.

The evaluator may compare these measured physiological data against goals or objectives previously entered into the system of the invention. These goals or objectives may arise from stored tables or data. In alternative embodiments, the objectives and goals may arise from third party input, e.g. clinician set objectives. Upon review of the data and determination of a response, the agent within the display system may initiate a query with the individual with the objective of enabling conveyance of the evaluator response to the individual. Such query may start with an inquiry by the evaluator regarding sleeping or rest, if the evaluator determines that the individual has not been resting adequately. In other instances, the evaluator may ask about lethargy and whether the individual has headaches, if there is an indication that the individual is dehydrated. These inquiries may be followed by suggestions to change behavior, e.g. try having an extra glass of juice two or three times a day, to perk up. Such advice may or may not directly link the suggested behavior to the measured physiological parameter, dependent upon evaluator determination of the most effective forms of communication for that individual.

The agent in the system may also provide activities that would in general support desired behavior as well as enable further queries by the individual regarding their well being. In certain embodiments, these queries may be relayed to a third party, e.g. clinician, or to other individuals using the system in general as a support group. Incentives for the individual to follow the suggestions of the agent may include discounts for local shops or activities or award points that other individuals might be able to see and compare themselves to.

### Example 4

### Cellular Phone Lifestyle Support

The device of the present invention may be substantially encompassed within a cellular phone or similar device. Portions of the device, e.g. specific sensor attachments, may be attached to the cellular phone in order to provide additional functionalities, if needed. In use, the individual would download the desired electronic media (software) from a remote data management system, e.g. download the application, and possibly purchase one or more necessary attachments, e.g. sensor modules, if these were not part of existing phone features. The individual would then initiate the program, and enter data necessary for algorithm function, e.g. age, gender, weight, height, waist dimension, etc. In addition, the user might enter in a desired goal or objective. The user would then select the form of electronic media involvement, e.g. role playing, direct scoring, etc, based upon the media type and user desires. The media would interact with the user through the cellular phone display and audio functionalities. The evaluator would be contained in part or in whole within the existing cellular phone circuitry and utilize one or more download software packages.

In use, the individual may activate the media, e.g. play the game, for a period of time. During those periods of time when not directly utilizing the media, the user may periodically take one or more physiological measurements to track progress towards the desired goal, e.g. by depressing a set of keystrokes or switch. The evaluator function would operate to record said data. In addition, the cellular phone may automatically record additional sensor data, e.g. motion or activity to enable subsequent assessment of movement, e.g. exercise, for use within the media. Upon reactivating the media, the evaluator would automatically award points to the user based upon one or more measured physiological data, either obtained during media operation or during the interval between media use. These points may then be used to motivate the individual directly, e.g. changing the level of play in the media, or be used as part of a reward system leading to intangible or tangible rewards, based on media type and/or user selection.

Other embodiments and applications of the invention are readily conceivable and are contained within the scope of the present invention. Accordingly, the scope of the present invention is not limited to those embodiments and applications described herein.

What is claimed is:

1. A system to aid in improving or maintaining the health of an individual comprised of:
at least one monitor enabling the measurement of at least one physiological parameter associated with the health status or change of health status of said individual;
at least one evaluator that contained substantially within at least one electronic device wherein said evaluator can receive physiological data from said monitor to determine the health status or change in health status of the individual;
at least one response to said determination being generated by at least one evaluator, based upon current, past, comparative, or stored physiological data;
and, at least one response being conveyed to at least one electronic media for conveyance to the individual for the purpose of improving the health of said individual.

2. The response of claim 1 wherein said response is forward looking in time, e.g. as an incentive.

3. The response of claim 1 wherein said response is retrospective, e.g. as a reward for meeting a physiological goal or objective.

4. The system of claim 1 where said monitor include sensors detection of body composition, fluid change, heart rate, or motion.

5. The system of claim 1 where said monitor is affixed to a region of the body through use of adhesives.

6. The system of claim 1 where said monitor substantially planar and flexible.

7. The system of claim 1 where said monitor communicates by wireless means to said electronic device.

8. The system of claim 1 where said evaluator utilizes a plurality of received physiological data to enable determination of change in health of said individual

9. The system of claim 1 where said physiological data corresponds to physiological measurements taken over a period of time while the individual is not directly engaged with the electronic media.

10. The system of claim 1 where said response is based at least in part on one or more reference tables of one or more physiological parameters.

11. The system of claim 1 where said response is reflective of a determined change in one or more physiological parameters over a period of time

12. The response of claim 1 where said response is in the form of a tangible or intangible reward.

13. The system of claim 1 where said response is intended to result in a change in the behavior of the individual that will lead to a change in one or more physiological parameters over a period of time.

14. The response of claim 12 where said response is in the form of an incentive.

15. The system of claim 1 where said electronic media is a computer based game.

16. The system of claim 15 where said game enables evaluator response to be delivered by an electronic agent.

17. The system of claim 15 where said response results in the change in one or more features of a computer based game, including change in capabilities of one or more avatars substantially controlled by the individual, or change in the virtual environment in which said avatar is located.

18. The system of claim 1 where said system is substantially incorporated within a cellular phone.

19. The system of claim 1 where said improvement of health relates to values provided to the evaluator by the user.

20. A system for improving the health of an individual utilizing:
at least one set of measured physiological data;
at least one evaluation of said measured data to ascertain health status of the individual;
and at least one response to improve the health of the individual conveyed to electronic media resulting in an adjustment of said media.

21. The method of claim 18 where said physiological data includes metabolic data regarding body composition, fluid status, motion or heart rate.
